(19) United States
(12) Patent Application Publication
(10) Pub. No.: US 2007/0014859 A1
(43) Pub. Date: Jan. 18, 2007

(54) Highly bioavailable oral delayed release dosage forms of O-desmethylvenlafaxine succinate

(75) Inventors: Syed M. Shah, East Hanover, NJ (US); Mahdi B. Fawzi, Morristown, NJ (US); Christopher Richard Diorio, Campbell Hal, NY (US)

Correspondence Address: HOWSON AND HOWSON;CATHY A. KODROFF, SUITE 210, 501 OFFICE CENTER DRIVE, FT WASHINGTON, PA, 19034, US

(73) Assignee: Wyeth, Madison, NJ (US)

(21) Appl. No.: 11/486,324

(22) Filed: Jul. 13, 2006

Related U.S. Application Data
(60) Provisional application No. 60/699,623, filed on Jul. 15, 2005.

(51) Int. Cl. A61K 9/22; A61K 9/26

(52) U.S. Cl. 424/468; 424/470

(57) ABSTRACT

An oral, highly bioavailable unit dosage form of O-desmethylvenlafaxine succinate (DVS) having a delayed release of at least about one hour and a sustained release over multiple hours to provide a total release of greater than about 85% within about 12 to about 14 hours is described. In one embodiment, the superbioavailable DVS composition has a delayed release of about two hours and a total release of greater than about 95% within about 12 to about 14 hours. Use of the formulation in treating depression and reducing the gastrointestinal side-effects of O-desmethylvenlafaxine (ODV) is also described.

### CROSS-REFERENCE TO RELATED APPLICATION

This application claims the benefit under 35 USC 119(e) of U.S. Provisional Patent Application No. 60/699,623, filed Jul. 15, 2005.

### BACKGROUND OF THE APPLICATION

The invention relates to an oral, highly bioavailable dosage form of O-desmethylvenlafaxine succinate, and to its use in treating depression and reducing the side-effects of O-desmethylvenlafaxine.

O-desmethylvenlafaxine (ODV), the major metabolite of venlafaxine, selectively blocks the reuptake of serotonin and norepinephrine. Klamerus, K. J. et al., “Introduction of the Composite Parameter to the Pharmacokinetics of Venlafaxine and its Active O-Desmethyl Metabolite”, J. Clin. Pharmacol. 32:716-724 (1992). O-desmethyl-venlafaxine, chemically named 1-[2-(dimethylamino)-1-(4-phenol)ethyl]-cyclohexanol, was exemplified as a fumarate salt in U.S. Pat. No. 4,535,186. However, the fumarate salt of O-desmethyl-venlafaxine has unsuitable physicochemical and permeability characteristics. O-desmethyl-venlafaxine is also exemplified as a free base in International Patent Publication No. WO 00/32555.

The succinate form of ODV has been described [U.S. Pat. No. 6,673,838]. The succinate monohydrate form of ODV has been incorporated into an extended release hydro-gel tablet, which reduces adverse effects such as nausea, vomiting, diarrhea, and abdominal pain. Formulations describing the use of hydroxypropyl methylcellulose (HPMC) as the hydrogel matrix have been described [WO 02/064543 A2].

However, the effects of the hydrogel formulation have been observed to be variable when the ODV hydrogel tablet is given with food.

### SUMMARY OF THE INVENTION

The present invention provides oral delayed release dosage units composed of ODV succinate, termed herein DVS, and an enteric coat in the range of about 10 to 20 wt % of the dosage unit. These oral delayed release dosage units enhance bioavailability, reduce undesirable side effects, and reduce variability in plasma.

Advantageously, in one embodiment, the compositions of the invention enhance the bioavailability of ODV succinate by deferring release of most of the ODV succinate until such time as the formulation is in the ileum and small intestine, while minimizing colonic release. Further, compositions described herein provide sustained release over a period of at least 8 hours, while providing at least about 85% total release within 12 hours of the oral dosage unit being taken orally.

These and other advantages of the invention will be readily apparent from the following detailed description of the invention.

### BRIEF DESCRIPTION OF THE FIGURES

FIG. 1 provides a chart showing the release profile of a superbioavailable 150 mg DVS oral dosage unit of the invention.

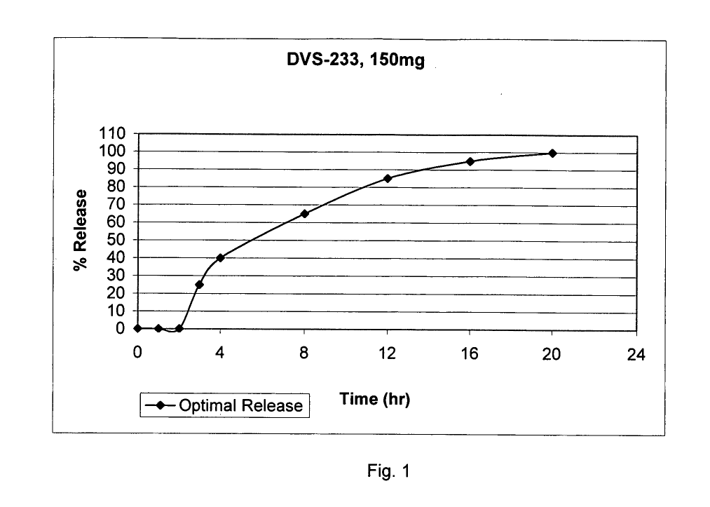

### DETAILED DESCRIPTION OF THE INVENTION

The present invention provides an oral, highly bioavailable, dosage unit of DVS. These sustained release formulations lower the incidence of side effects, including nausea, emesis, and irritable bowel syndrome. Without wishing to be bound by theory, it is believed that these side-effects are avoided by by-passing release in the upper GI tract and providing release in the lower GI tract. Further, use of the superbioavailable DVS provided herein is believed to result in reduced patient variability in plasma exposure.

Advantageously, in one embodiment, the superbioavailable DVS formulation of the invention comprises DVS in an oral dosage unit having a delayed release of at least about one hour and a sustained release over multiple hours to provide a total release of greater than about 85% within about 12 to about 14 hours. In one embodiment, the superbioavailable sustained release DVS formulation has a delayed release of about two hours and a total release of greater than about 95% within about 12 to about 14 hours.

In one embodiment, the DVS oral dosing units of the invention are composed, at a minimum, of a core containing DVS, and one or more pharmaceutically acceptable excipients. Suitably, the core contains about 40 wt % to about 60 wt % DVS, about 45 to 55 wt %, or about 47 to 52 wt %, of the total oral dosing unit. The core containing the DVS may be in a sustained release formulation or other suitable cores as are described in greater detail below may be selected. In one embodiment, a delay release coat and/or an enteric coat are provided over the core.

The delay release coat and/or an enteric coat (rate-controlling film) can be applied to the DVS core directly, or there may be intermediate coating layers located between the DVS core and any over coats. Optionally, a further seal or top coat may be located outside the enteric coat.

DVS is prepared as described in U.S. Pat. No. 6,673,838, which is incorporated by reference herein. In other embodiments, the DVS can range from about 20% w/w to about 75 wt % w/w, 25 wt % to about 50 wt %, from about 30 wt % to about 45 wt %, or from about 35 wt % to about 55 wt %, based upon 100% weight of the core. Suitably, the DVS can range from about 10% w/w to about 70% w/w of the total oral dosage unit, and preferably, about 40 to about 60 wt %, and more preferably, about 50 to about 55 wt % of the total weight of the oral dosage unit.

In one embodiment, the core contains about 25 wt % to about 30 wt % microcrystalline cellulose. In other embodiments, the core may contain another binder or additional binders, or further excipients such as diluents, fillers, glidants, anti-adherents, and adjuvants to provide a total amount of excipients in the core of about 25 wt % to about 80 wt % w/w of the core.

For example, when present, one or more binder/fillers and/or diluents can each be present in an amount of about 15% w/w to about 80% w/w, or about 20% w/w to about 70% w/w, or about 25% w/w to about 45% w/w, or about 30% w/w to about 42% w/w of the uncoated dosage form. The total amount of a pH adjuster in the formulation can range from about 0.1% w/w to about 10% w/w of the core, or about 1% w/w to about 8% w/w, or about 3% w/w to about 7% w/w. However, these percentages can be adjusted as needed or desired by one of skill in the art.

In one embodiment, the filler/binder is water insoluble. The filler/binder may be selected from among known fillers/binders, including, e.g., cellulose, and povidone, among others. In one embodiment, the filler/binder is selected from among microcrystalline cellulose, crospovidone, and mixtures thereof. Other suitable fillers/binders, including those that are water soluble or partially water soluble may be used in combination with water insoluble fillers/binders, as needed.

Suitable pH adjusters include, e.g., sodium carbonate, sodium bicarbonate, potassium carbonate, lithium carbonate, among others. Still other suitable components will be readily apparent to one of skill in the art.

In one embodiment, the DVS core is provided with further layers that provide a sustained release formulation which contains rate-controlling components. Typically, such rate controlling components are rate controlling polymers selected from among hydrophilic polymers and inert plasticized polymers. Suitable rate controlling hydrophilic polymers include, without limitation, polyvinyl alcohol (PVA), hypomellose and mixtures thereof. Examples of suitable insoluble or inert “plastic” polymers include, without limitation, one or more polymethacrylates (i.e., Eudragit® polymer). Other suitable rate-controlling polymer materials include, e.g., hydroxyalkyl celluloses, poly(ethylene) oxides, alkyl celluloses, carboxymethyl celluloses, hydrophilic cellulose derivatives, and polyethylene glycol.

Thus, in one embodiment, the formulation of the invention contains one or more coatings over the DVS core. In still other embodiments, the core can contain a non-functional seal coating (i.e., a coat which does not affect release rate) and a functional second coating.

In one embodiment, an initial seal coat can be applied directly to the core. Although the components of this seal coat can be modified by one of skill in the art, the seal coat may be selected from among suitable polymers such as hydroxypropyl methylcellulose (HPMC), ethylcellulose, polyvinyl alcohol, and combinations thereof, optionally containing plasticizers and other desirable components. A particularly suitable seal coat contains HPMC. For example, a suitable seal coat can be applied as a HPMC solution at a concentration of about 3% w/w to 25% w/w, and preferably 5% w/w to about 7.5% w/w.

The initial seal coat can be applied on a fluid bed coater, e.g., by spraying. In one embodiment, an Aeromatic Strea™ fluid bed apparatus is fitted with a Wurster column and bottom spray nozzle system. Approximately 200 grams of the dried pellet cores are charged into the unit. The Opadry® Clear seal coat is applied with an inlet temperature of approximately 50° C. to 60° C., a coating solution spray rate of 5 to 10 grams/minute, atomization pressure of 1 to 2 bar. The desired product temperature is 35° C. to 45° C., and preferably 38° C. to 43° C.

Upon drying, under suitable conditions, the initial seal coat is in the range of about 1% w/w to about 3% w/w, or about 2% w/w, of the uncoated core. In another embodiment, a commercially available seal coat containing HPMC, among other inert components, is utilized. One such commercially available seal coat is Opadry® Clear (Colorcon, Inc.).

In one embodiment, the oral dosage unit contains a further release or “delay” coating layer. This release coating layer may be applied over an initial seal coat or directly over a core.

In one embodiment, the release coat is a controlled release coating layer which contains an ethylcellulose-based product. An example of one suitable ethylcellulose-based product is an aqueous ethylcellulose dispersion (25% solids). One such product is commercially available as Surelease® product (Colorcon, Inc.). In one embodiment, a solution of an aqueous ethylcellulose (25% solids) dispersion of about 3% w/w to about 25% w/w, and preferably about 3% to about 7%, or about 5% w/w, is applied to the core. In another embodiment, the controlled release coat contains both an ethylcellulose-based product and hypomellose. Optionally, hypomellose, e.g., in an amount of about 5 to 15% by weight, and preferably, about 10% by weight, is mixed with the ethylcellulose dispersion, to form the coat solution. Thus, such the ethylcellulose may be about 85% to about 95%, by weight, or in embodiment, about 90% by weight, of the coat solution. Upon drying under suitable conditions, the total controlled release coat is in the range of about 2% to about 5%, or about 3% to about 4% w/w of the uncoated or initially-coated core.

In one embodiment, the oral dosage unit contains an enteric coat, which can provide an initial “delay”. In certain embodiments, the enteric coat may delay release for as much as about 30 minutes to two hours. The enteric coat may be applied over the controlled release coat, over an initial seal coat, or directly over a core.

The enteric coat may contain, e.g., polymethacrylates, hypomellose, and ethylcellulose, or a combination thereof.

In one embodiment, the enteric coat contains a product which is a copolymer of methacrylic acid and methacrylates, such as the commercially available Eudragit® L 30 K55 (Röhm GmbH & Co. KG). Suitably, this enteric coat is applied such that it coats the core in an amount of about 10 wt % to 20 wt %, or about 12 wt % to about 17 wt % , or about 15.5 wt % to 16.5 wt % of the uncoated or initially-coated core. In one embodiment, the enteric coat is composed of a Eudragit® L30D-55 copolymer (Röhm GmbH & Co. KG), talc, triethyl citrate, and water. More particularly, the enteric coating may contain about 7 wt % to about 9 wt % of a 30 wt % dispersion of Eudragit® L 30 D55 coating; about 4 wt % to about 5 wt % /w talc, about 0.7 wt % to about 1 wt % triethyl citrate; a pH adjuster such as sodium hydroxide and water.

The enteric coat can be applied directly to the uncoated spheroid core, i e., the uncoated core, or may be applied over an initial seal coat. The enteric coat, as described above, is typically applied on a fluid bed coater. In one embodiment, Surelease® aqueous ethylcellulose dispersion (25% solids) is applied in a similar fashion as the seal coat. After the ethylcellulose coat is applied, the pellets are dried for an additional 5 to 10 minutes. They are then removed and screened through a mesh screen to remove agglomerates and oversize particles.

In one embodiment, a final seal coat is applied over the enteric coat and, optionally, talc is utilized as a final step prior to filling the DVS formulations into a suitable packaging unit. Suitably, this final seal coat is composed of HPMC and water, upon drying, is less than about 1 wt % of the total, coated oral dosage unit.

The formulations described herein can be prepared using the techniques described herein, as well as methods known to those of skill in the art.

II. Formulations/Kits/Methods of delivery

In another embodiment, the present invention provides products containing the DVS formulations of the invention.

In one embodiment, the DVS formulations are packaged for use by the patient or his caregiver. For example, the formulations can be packaged in a foil or other suitable package and is suitable for mixing into a food product (e.g., applesauce or the like) or into a drink for consumption by the patient.

In another embodiment, the DVS formulations are suspended in a physiologically compatible suspending liquid. For oral liquid pharmaceutical compositions, pharmaceutical carriers and excipients can include, but are not limited to water, glycols, oils, alcohols, flavoring agents, preservatives, coloring agents, and the like.

In yet another embodiment, the DVS formulations are filled in capsules, caplets or the like for oral delivery.

In another embodiment, the present invention provides for the use of the formulations described herein in the preparation of medicaments, including but not limited to medicaments useful in the treatment of depression, gastrointestinal side-effects of venlafaxine in a subject undergoing treatment therewith, and irritable bowel syndrome.

In another embodiment, the present invention provides for the use of the formulations described herein in the preparation of medicaments for delivery to a pediatric or geriatric patient.

In other embodiments, the present invention provides for the use of the formulations described herein in the preparation of dosing units, including but not limited to dosing units for oral, transdermal, or mucosal administration.

Also encompassed by one embodiment of the invention are pharmaceutical packs and kits comprising a container, such as a foil package or other suitable container, having a formulation described herein in unit dosage form.

In still a further embodiment, the invention provides method of treating a subject in need thereof by administering an effective dose of the formulations of the invention. The formulations of the invention are useful in treatment of depression, anxiety, panic disorder, generalized anxiety disorder, post traumatic stress disorder, premenstrual dysphoric disorder, fibromyalgia, agorophobia, attention deficit disorder, obsessive compulsory disorder, social anxiety disorder, autism, schizophrenia, obesity, anorexia nervosa, bulimia nervosa, Gilles de la Tourette Syndrome, vasomotor flushing, cocaine and alcohol addiction, sexual dysfunction, borderline personality disorder, chronic fatigue syndrome, urinary incontinence, pain, Shy Drager syndrome, Raynaud's syndrome, Parkinson's disease, and epilepsy. These formulations are also useful for enhancing cognition or treating cognitive impairment in a patient, cessation of smoking or other tobacco uses in a patient, treating hypothalamic amenorrhea in a depressed or non-depressed human female, lowering the incidence of nausea, vomiting, diarrhea, abdominal pain, headache, vaso-vagal malaise, or trismus resulting from the oral administration of O-desmethylvenlafaxine succinate.

Suitably, the formulations of the invention can reduce the gastrointestinal side-effects of venlafaxine in a subject undergoing treatment therewith comprising administering to the patent a formulation of the invention. Without wishing to be bound by theory, it is anticipated that providing an oral dosage unit as described herein having a core of the sustained release formulation described herein will result in a composition of the invention which enhances the bioavailability of DVS by deferring release of most of the DVS until such time as the formulation is in the ileum and small intestine, while minimizing colonic release. Such a composition is anticipated to have a delayed release of at least about one hour and a sustained release over multiple hours to provide a total release of greater than about 85% within about 12 to about 14 hours. It is also anticipated that providing a pelleted oral dosage unit of the invention will provide low levels of variability (if any) in plasma exposure and will provide low incidences of nausea and associated side effects.

An effective amount of the oral dosage units of the invention is an amount sufficient to prevent, inhibit, or alleviate one or more symptoms of the aforementioned conditions. The dosage amount useful to treat, prevent, inhibit or alleviate each of the aforementioned conditions will vary with the severity of the condition to be treated and the route of administration. The dose, and dose frequency will also vary according to age, body weight, response and past medical history of the individual human patient. In general the recommended daily dose range for the conditions described herein lies within the range of 10 mg to about 1000 mg ODV per day and more preferably within the range of about 37.5 mg to about 300 mg/day and still more preferably from about 50 mg to about 200 mg/day. In other embodiments of the invention the dosage will range from about 30 mg to about 90 mg/day. Dosage is described in terms of the free base (ODV) and is adjusted accordingly for the succinate salt (DVS). For example, a 100 mg ODV strength oral dosage unit of the formulation typically contains about 152 mg of DVS-233. In another example, a 150 mg ODV strength oral dosage unit of the invention typically contains about 228 mg of DVS-233. In managing the patient, is generally preferred that the therapy be initiated at a lower dose and increased if necessary. Dosages for non-human patients can be adjusted accordingly by one skilled in the art.

DVS may also be provided in combination with other active agents including, e.g., venlafaxine. The dosage of venlafaxine is preferably about 75 mg to about 350 mg/day and more preferably about 75 mg to about 225 mg/day. Still more preferably the dosage of venlafaxine is about 75 mg to about 150 mg/day. The ratio of DVS will vary from patient to patient depending upon a patient's response rate, but generally will be at least 6:1 ODV salt to venlafaxine. Venlafaxine or another active agent delivered in a regimen with the oral dosage unit of the invention may be formulated together with the oral dosage unit of the invention, or delivered separately.

Any suitable route of administration can be employed for providing the patient with an effective amount of DVS. For example, oral, mucosal (e.g., nasal, sublingual, buccal, rectal or vaginal), parental (e.g., intravenous or intramuscular), transdermal, and subcutaneous routes can be employed. Preferred routes of administration include oral, transdermal and mucosal.

DVS can be combined with a pharmaceutical carrier or excipient (e.g., pharmaceutically acceptable carriers and excipients) according to conventional pharmaceutical compounding technique to form a pharmaceutical composition or dosage form. Suitable pharmaceutically acceptable carriers and excipients include, but are not limited to, those described in Remington's, The Science and Practice of Pharmacy, (Gennaro, A. R., ed., 19^{th} edition, 1995, Mack Pub. Co.) which is herein incorporated by reference. The phrase “pharmaceutically acceptable” refers to additives or compositions that are physiologically tolerable and do not typically produce an allergic or similar untoward reaction, such as gastric upset, dizziness and the like, when administered to an animal, such as a mammal (e.g., a human).

Oral solid pharmaceutical compositions may include, but are not limited to starches, sugars, microcrystalline cellulose, diluents, granulating agents, lubricants, binders and disintegrating agents. The pharmaceutical composition and dosage form may also include venlafaxine or a salt thereof as discussed above.

The following examples illustrate exemplary dosage forms of the invention, and the use thereof. These examples are not a limitation on the present invention.

### EXAMPLE 1

### 2% Surelease (Ethylcellulose Dispersion)

| | mg/capsule (150 mg ODV |
|---|---|
| Ingredient | dosage) |
| Pellet Core: | |
| DVS-233 | 227.62 |
| Microcrystalline cellulose | 97.55 |
| Seal Coat: | |
| Opadry Clear | 6.50 |
| Release Coat: | |
| Ethylcellulose dispersion (NF) | 6.50 |
| Enteric Coat: | |
| Eudragit L30-D55 | 71.77 |
| Triethyl Citrate | 2.15 |
| Sodium Hydroxide | 3.23 |
| Talc | 10.64 |
| Water* | NA |

*Does not appear in final formula

### EXAMPLE 2

### 3% Surelease (Ethylcellulose Dispersion)

| | mg/capsule (150 mg ODV |
|---|---|
| Ingredient | dosage) |
| Pellet Core: | |
| DVS-233 | 227.62 |
| Microcrystalline cellulose | 97.55 |
| Seal Coat: | |
| Opadry Clear | 6.50 |
| Release Coat: | |
| Ethylcellulose dispersion (NF) | 9.75 |
| Enteric Coat: | |
| Eudragit L30-D55 | 71.77 |
| Triethyl Citrate | 2.15 |
| Sodium Hydroxide | 3.23 |
| Talc | 10.64 |
| Water* | NA |

*Does not appear in final formula

### EXAMPLE 3

### Enteric Coated Capsule with Hypromellose/Microcrystalline Cellulose Pellet Core

| | mg/capsule (150 mg ODV |
|---|---|
| Ingredient | dosage) |
| Pellet Core: | |
| DVS-233 | 227.62 |
| Microcrystalline cellulose | 97.55 |
| Hypromellose | 65.0 |
| Seal Coat: | |
| Opadry Clear | 6.50 |
| Enteric Coat: | |
| Eudragit L30-D55 | 71.77 |
| Triethyl Citrate | 2.15 |
| Sodium Hydroxide | 3.23 |
| Talc | 10.64 |
| Water* | NA |

*Does not appear in final formula

This formula is anticipated to have a release of greater than 85% of its content, in vivo, within 12 hours of the product being taken orally after a 2 hour lag period and with about 100% release within 20 hours.

### EXAMPLE 4

### Superbioavailable Formulations With Hypromellose/Microcrystalline Cellulose in Core and Without Enteric Coat

A. Capsule Dosage Unit with Pellet Core and Delay Coat

| | mg/capsule (150 mg ODV |
|---|---|
| Ingredient | dosage) |
| Pellet Core: | |
| DVS-233 | 227.62 |
| Microcrystalline cellulose | 97.55 |
| Hypromellose | 65.0 |
| Seal Coat: | |
| Opadry Clear | 6.50 |
| “Delay” Coat: | |
| Surelease ® ethylcellulose | 27.0 |
| dispersion | |
| Hypomellose | 3.0 |
| Water* | NA |

*Does not appear in final formula

B. Tablet Dosage Unit with Delay Coat

| | mg/tablet (150 mg ODV |
|---|---|
| Ingredient | dosage) |
| Tablet Core: | |
| DVS-233 | 227.62 |
| Hypromellose | 135.00 |
| Microcrystalline cellulose | 62.00 |
| Talc | 18.00 |
| magnesium stearate | 7.00 |
| “Delay” Coat: | |
| Surelease ® ethylcellulose | 27.0 |
| dispersion | |
| Hypomellose | 3.0 |
| Water* | NA |

C. Tablet Core with Enteric Coat

| | mg/tablet (150 mg ODV |
|---|---|
| Ingredient | dosage) |
| Tablet Core: | |
| DVS-233 | 227.62 |
| Hypromellose | 135.00 |
| Microcrystalline cellulose | 62.00 |
| Talc | 18.00 |
| magnesium stearate | 7.00 |
| Enteric (“delay”) Coat: | |
| Eudragit L30-D55 | 71.77 |
| Triethyl Citrate | 2.15 |
| Sodium Hydroxide | 3.23 |
| Talc | 10.64 |

### EXAMPLE 5

### Bioavailability Studies

The compositions of the invention are designed to enhance the bioavailability of DVS by deferring release of most of the DVS until such time as the formulation is in the ileum and small intestine, while minimizing colonic release.

This example describes a study performed to assess the absolute bioavailability of an exemplary sustained release desvenlafaxine succinate (DVS-SR) formulation and the pharmacokinetics of desvenlafaxine (DVS) in healthy subjects.

In one embodiment the present invention provides an oral dosage unit composed of a core of the following sustained release DVS formulation having an enteric coat such as is described in Example 4C. In another embodiment, the invention provides an oral dosage unit comprising a delay release coat over a core composed of the following sustained release DVS formulation.

100 MG ODV STRENGTH DVS-233 SR TABLETS

100 mg

| Ingredient | mg/Tablet | % w/w |
|---|---|---|
| DVS-233 | 151.74 | 44.63 |
| Succinate Monohydrate | (100.0)^{a} | |
| Hydroxypropyl | 170.00 | 50.00 |
| Methylcellulose 2208 Usp, | | |
| 100,000 Cr (Methocel, K100 | | |
| M Premium Cr) | | |
| (Intra-Granular) | | |
| Microcrystalline Cellulose Nf | 7.20 | 2.11 |
| (Avicel Ph200) | | |
| Talc Usp, Luzenac Pharma | 7.65 | 2.25 |
| Magnesium Stearate Nf/Ep | 3.40 | 1.00 |
| (Vegetable Grade) | | |
| Totals | 340 | 100 |
| PURIFIED WATER | | |
| USP/BP/Ep^{b} | | |

^{a}As base (Theory = 65.9% as base).
^{b}Used in processing - does not appear in final product.

In this single-dose, open label, two period crossover study, subjects were randomized to receive either a 1×100 mg oral tablet of DVS-SR as described in the table above or a single 50 mg/l hr intravenous infusion of desvenlafaxine succinate (DVS) in 0.9% saline in each period. Plasma was assayed for the total racemic mixture (R+S) and ratio (R/S) of DV. The absolute bioavailability was calculated from oral and IV AUC values of the racemic mixture of DVS.

A total of 14 subjects were enrolled and completed the study. DVS-SR was generally well tolerated. There were no clinically important changes in routine laboratory tests, vital signs measurements, and ECGs. The 50 mg IV formulation had a higher Cₘₐₓ(232 ng/mL) than the 100 mg oral formulation (160 ng/mL). The half-lives were similar, ranging from 14-15 hours, and the 100 mg oral formulation of DVS-SR had a higher overall exposure (AUCₒᵣₐₗ 3996 vs. AUC_{IV} 2443 ng*h/mL). The absolute bioavailability of the oral formulation was 80.5%. The R and S enantiomers were approximately equivalent to each other throughout the concentration profiles for both the IV and oral formulations.

DVS-SR provided good oral bioavailability (80.5%) and an evenly balanced enantiomeric ratio.

### EXAMPLE 6

### Bioavailability Study in Canines

The DVS-233 formula of Example 1 was formulated in a capsule. This “prototype” DVS-233 capsule was compared in a bioavailability study in dogs to a DVS-233 SR tablet having a formulation of the table in Example 5, which lacks a delay or enteric coating. Six female dogs were assigned to this study. The prototype capsule and SR tablet were administered as a single oral dose to each dog in a crossover design, approximately 30 minutes after being fed. Blood samples were drawn at 0 (predose), 0.5, 1, 2, 3, 4, 6, 8, 12 and 24 hours after dosing, plasma was separated and assayed for O-desmethylvenlafaxine (ODV) content. The pharmacokinetic parameter (AUC_{0-∞}) was determined for each dog and descriptive statistics were calculated. The prototype capsule provided approximately 20% higher total exposure indicating higher bioavailability.

The present invention is not to be limited in scope by the specific embodiments described herein. Various modifications to these embodiments will be obvious to one of skill in the art from the description. Such modifications fall within the scope of the appended claims.

Patents, patent applications, publications, procedures and the like are cited throughout the application. The disclosures of these documents are incorporated by reference herein in their entireties. To the extent that a conflict may exist between the specification and a reference, the language of the disclosure made herein controls.

1. A superbioavailable DVS (O-desmethylvenlafaxine succinate) sustained release composition comprising a core containing at least DVS and a water insoluble filler in an oral dosage unit having a delayed release of at least about one hour and a sustained release over multiple hours to provide a total release of greater than about 85% within about 12 to about 14 hours.

2. The superbioavailable DVS composition according to claim 1, having a delayed release of about two hours and a total release of greater than about 95% within about 12 to about 14 hours.

3. The superbioavailable DVS composition according to claim 1, wherein said oral dosage unit further comprises a controlled release coat comprising ethylcellulose.

4. The superbioavailable DVS composition according to claim 3, wherein said controlled release coat further comprises about 10% by weight hypomellose, based on the weight of the controlled release coat.

5. The superbioavailable DVS composition according to claim 3, wherein said controlled release coat comprises about 90% by weight of an ethylcellulose dispersion, based on the weight of the controlled release coat.

6. The superbioavailable DVS composition according to claim 3, wherein said controlled release coat consists of about 5 to 20% by weight of the oral dosage unit.

7. The superbioavailable DVS composition according to claim 3, wherein the core comprises DVS and microcrystalline cellulose, and the oral dosage unit further comprises a controlled release coat and an enteric coat.

8. The superbioavailable DVS composition according to claim 3, further comprising a seal coat between the core and the controlled release coat.

9. The superbioavailable DVS composition according to claim 1, wherein the core comprises DVS, microcrystalline cellulose and a matrix forming polymer, and the oral dosage unit further comprises an enteric coat.

10. The superbioavailable DVS composition according to claim 9, wherein the matrix forming polymer is hypomellose.

11. The superbioavailable DVS composition according to claim 9, wherein the core further comprises talc.

12. The superbioavailable DVS composition according to claim 1, wherein the core comprises:

| | |
|---|---|
| DVS | about 44 to 46 wt % |
| Hypomellose | about 12 to 14 wt % |
| Microcrystalline cellulose | about 21 to 22 wt % |
| Talc | about 2 to 4 wt % |
| Lubricant | about 1%, of the total oral dosage |

unit.

13. The superbioavailable DVS composition according to claim 1, wherein DVS comprises 40 to 60 wt % of the oral dosage unit.

14. The superbioavailable DVS composition according to claim 1, wherein the oral dosage unit comprises ODV in the range of 37.5 mg to 300 mg.

15. The superbioavailable DVS composition according to claim 14 wherein the oral dosage unit is a 200 mg ODV strength dosage unit.

16. The superbioavailable DVS composition according to claim 14, wherein the oral dosage unit is a 150 mg ODV strength dosage unit.

17. The superbioavailable DVS composition according to claim 14, wherein the oral dosage unit is a 100 mg ODV strength dosage unit.

18. The superbioavailable DVS composition according to claim 14, wherein the oral dosage unit is a 50 mg ODV strength dosage unit.

19. The superbioavailable DVS composition according to claim 1, further comprising an the enteric coat comprises 10 to 20% by weight of the oral dosage unit.

20. The superbioavailable DVS composition according to claim 19, wherein the enteric coat comprises a methacrylic acid co-polymer, triethyl citrate, sodium hydroxide and talc.

21. The superbioavailable DVS composition according to claim 19, wherein the enteric coat comprises

| | |
|---|---|
| Eudragit L30D-55 | about 7 to 9 wt % |
| Triethyl citrate | about 0.7 to 1 wt % |
| Sodium hydroxide | about 1 to 1.5 wt % |
| Talc | about 4 to 5 wt %, of the oral dosage unit. |

22. A method for treating depression in a subject in need thereof, comprising administering to the patient a composition comprising a superbioavailable DVS composition according to claim 1.

23. A method for reducing the gastrointestinal side-effects of desvenlafaxine in a subject undergoing treatment therewith comprising administering to the patent a composition comprising a superbioavailable DVS composition according to claim 1.

24. The method according to claim 23, wherein the gastrointestinal side-effects are nausea and vomiting.

25. A pharmaceutical pack comprising a container having a superbioavailable DVS composition according to claim 1.
